(19) United States
(12) Patent Application Publication
(10) Pub. No.: US 2009/0248082 A1
(43) Pub. Date: Oct. 1, 2009

(54) SURGICAL SYSTEMS AND METHODS FOR JOINT FIXATION

(76) Inventors: DAVID CROOK, Mineola, TX (US); Javier Garcia-Bengochea, Jacksonville, FL (US); Trace Cawley, Boca Raton, FL (US)

Correspondence Address: Clements Bernard PLLC, 1901 Roxborough Road, Suite 250, Charlotte, NC, 28211, US

(21) Appl. No.: 12/397,884

(22) Filed: Mar. 4, 2009

Related U.S. Application Data
(60) Provisional application No. 61/033,625, filed on Mar. 4, 2008.

(51) Int. Cl. A61B 17/70

(52) U.S. Cl. 606/263; 606/246

(57) ABSTRACT

The present disclosure provides surgical systems and method for joint fixation device with a self-locking mechanism to provide stabilization and immobilization of a joint, such as a facet joint or the like. The present invention includes an elongated fixation mechanism that is placed through an opening in a joint and inserted into a retention mechanism. The retention mechanism is operable to provide a self-locking mechanism to the elongated fixation mechanism to provide fixation of the joint. In an exemplary embodiment, the present invention can be utilized by a surgeon through a minimally-invasive surgical procedure to provide facet joint fixation.

### CROSS-REFERENCE TO RELATED APPLICATION(S)

The present non-provisional patent application claims priority to U.S. Provisional Patent Application Ser. No. 61/033,625, filed Mar. 4, 2008, and entitled “SYSTEMS AND METHODS FOR SPINAL FACET JOINT FIXATION WITH A SELF-TYING LACE,” the contents of which are incorporated in-full by reference herein.

### FIELD OF THE INVENTION

The present invention relates generally to surgical fixation devices and methods. More particularly, the present invention provides surgical systems and methods for a joint fixation device with a self-locking mechanism to provide stabilization and immobilization of a joint, such as a facet joint or the like.

### BACKGROUND OF THE INVENTION

When confronted with various spinal diseases and injuries, it is often desirable for a spinal surgeon to perform an interbody fusion or the like, whereby adjacent vertebrae are fused together using a bone graft and/or an implantable device, or otherwise immobilize/stabilize a portion of the spine of a patient. Typically, in the interbody fusion case, the adjacent vertebrae are immobilized while the bone graft is allowed to “take,” for example, using a conventional pedicle screw system, a plate system, or the like. Such a pedicle screw system includes a plurality of pedicle screws that are anchored to adjacent levels of the spine and connected with stabilizing rods or the like. Such a plate system includes a plate that is anchored to adjacent levels of the spine and, optionally, connected to the implantable device.

Facet joints are associated with each vertebra in the human spine. Four facet joints are associated with each vertebra. Four facet joints in pairs interlock with adjacent vertebrae and provide spine stability. The facet joints can be damaged by either a traumatic injury or degenerative processes. The damage to facet joints can result in the eventual degeneration of the facet joints, resulting in what is commonly referred to as a “pinched nerve.” This can also be referred to as nerve compression or nerve impingement, resulting in pain, misaligned anatomy, and possible loss of mobility.

A conventional treatment of the facet joints is spinal stabilization, commonly referred to as intervertebral stabilization. Intervertebral stabilization is designed to prevent relative motion between the vertebrae of the spine. Reducing the movement of the vertebrae ceases or at least decreases the resulting pain. Stabilization of the fusion site is advantageous in the early post-operation period. Similar to fracture healing, new bone is overlayed between the vertebral bodies, using the graft material as a lattice. Using cages as stand alone devices has met with limited success for the reason that immediate stabilization is not always guaranteed. For this reason, supplemental fixation is becoming more commonplace.

Another attractive option when treating various spinal diseases and injuries is to immobilize/stabilize the associated facet joint(s) using one or more facet bolts or the like. In order to accomplish this, the superior and inferior facets to be joined must be aligned and securely held during drilling and bolt placement, for example. It is also desirable that they are compressed either before or during drilling and bolt placement. This can be a tricky process, which is never desirable during a surgical procedure.

Existing facet joint fixation systems and methods utilize screws as a means of creating a posterior tension band to support an Interbody fusion. There is an ongoing need to provide for joint fixation that can be delivered simply, accurately, and quickly, while providing performance that is superior or equal to that of screw fixation.

### BRIEF SUMMARY OF THE INVENTION

In various exemplary embodiments, the present invention provides surgical systems and methods for a joint fixation device with a self-locking mechanism to provide stabilization and immobilization of a joint, such as a facet joint or the like. The present invention includes an elongated fixation mechanism that is placed through an opening in a joint and inserted into a retention mechanism. The retention mechanism is operable to provide a self-locking mechanism to the elongated fixation mechanism to provide fixation of the joint. In an exemplary embodiment, the present invention can be utilized by a surgeon through a minimally-invasive surgical procedure to provide facet joint fixation.

In an exemplary embodiment of the present invention, a surgical joint fixation system includes a retention mechanism defining an opening; and an elongated fixation member with a first end and a second end, wherein the first end is disposed to the retention mechanism and the second end is selectively disposed through a hole in a joint and through the opening in the retention mechanism. The surgical joint fixation system further includes a locking mechanism between the retention mechanism and the elongated fixation member operable to lock the second end to the retention mechanism. The locking mechanism optionally includes a plurality of notches disposed along a portion of the length of the elongated fixation member and a latch disposed to the opening of the retention mechanism. The surgical joint fixation system further includes a load bearing element disposed between the joint and the elongated fixation member, wherein the load bearing element is operable to move a load formed by the elongated fixation member across the joint. The load bearing element wraps around the joint and engages an opening of the hole in the joint. The surgical joint fixation system further includes a guide operable to route the elongated fixation member from the retention mechanism to the hole and from the hole back to the opening of the retention mechanism. The guide is removed following insertion of the second end of the elongated fixation member into the opening of the retention mechanism. The elongated fixation member is pulled through the opening to provide a ratchet-like tension to the joint. Optionally, a surgeon utilizes the surgical joint fixation system with a minimally invasive surgical procedure to provide immobilization and stabilization of a facet joint.

In another exemplary embodiment of the present invention, a facet joint fixation system includes a retention mechanism defining an opening with a locking device; an elongated fixation member fixedly attached to the retention mechanism at one end of the elongated fixation member, wherein the elongated fixation member includes a plurality of locking points substantially extending from the one end of the elongated fixation member to another end of the elongated fixation member, wherein the plurality of locking points are operable to lock with the locking device when the another end of the elongated fixation member is positioned and pulled through the opening of the retention mechanism; a guide operable to guide the elongated fixation member to a hole through adjacent facets; and a load bearing element placed between the elongated fixation member and the adjacent facets. The guide is removed following insertion of the elongated fixation member into the opening of the retention mechanism. The elongated fixation member is pulled through the opening of the retention mechanism to provide a ratchet-like tension to the facets. Optionally, a surgeon utilizes the facet joint fixation system with a minimally invasive surgical procedure to provide immobilization and stabilization of the facet joint.

In yet another exemplary embodiment of the present invention, a joint fixation method includes forming a hole through a joint; routing an elongated fixation member through the formed hole, wherein the elongated fixation member is fixedly attached to a retention member at one end; routing the elongated fixation member from the formed hole back to the retention member; inserting the elongated fixation member into an opening in the retention member, wherein the opening includes a locking mechanism; and adjusting the elongated fixation member to a tension for fixing the joint with the locking mechanism. The routing steps utilize a guide to guide the elongated fixation member. The joint fixation method further includes placing a load bearing element between the elongated fixation member and the joint before adjusting the elongated fixation member. The adjusting step further includes forming the load bearing element to the joint and to the hole in the joint. The adjusting step further includes automatically locking of the elongated fixation member with the retention member through a ratchet mechanism. The ratchet mechanism includes the plurality of locks on the elongated fixation member each operable to lock to a lock on the retention member.

### BRIEF DESCRIPTION OF THE DRAWINGS

The present invention is illustrated and described herein with reference to the various drawings, in which like reference numbers denote like method steps and/or system components, respectively, and in which:

FIG. 1 is a diagram of joint fixation system for stabilizing a joint in a pre-deployed configuration according to an exemplary embodiment of the present invention;

FIG. 2 is a diagram of a joint fixation system for stabilizing a joint in a deployed configuration according to an exemplary embodiment of the present invention; and

FIG. 3 is a flowchart of a mechanism for fixing joints according to an exemplary embodiment of the present invention.

### DETAILED DESCRIPTION OF THE INVENTION

In various exemplary embodiments, the present invention provides surgical systems and methods for a joint fixation device with a self-locking mechanism. The present invention includes an elongated fixation mechanism that is placed through an opening in a joint and inserted into a retention mechanism. The retention mechanism is operable to provide a self-locking mechanism to the elongated fixation mechanism to provide fixation of the joint. In an exemplary embodiment, the present invention can be utilized by a surgeon through a minimally-invasive surgical procedure to provide facet joint fixation such as fixing a superior facet and an inferior facet of a facet joint.

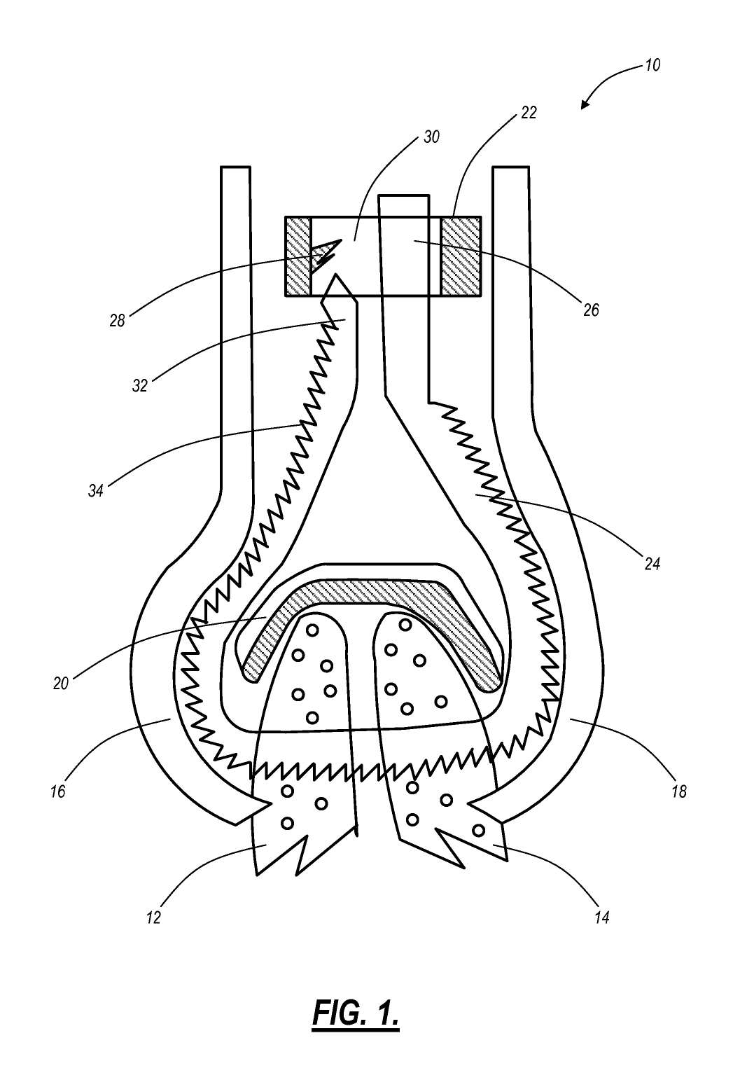

Referring to FIG. 1, a joint fixation system 10 for fixing, stabilizing, and/or immobilizing a joint is illustrated according to an exemplary embodiment of the present invention. For example, the joint can include a facet joint formed by a superior facet 12 and an inferior facet 14. The joint fixation system 10 can also be utilized with other joints. The joint fixation system 10 includes guides 16, 18, a load bearing element 20, a retention member 22, and an elongated fixation member 24. FIG. 1 illustrates the joint fixation system 10 engaged through the facets 12, 14 prior to locking the system 10. Accordingly, the elongated fixation member 24 is disposed through both facets 12, 14 through a hole formed through both the facets 12, 14. The joint fixation system 10 provides stabilization and immobilization of the facet joint formed by the facets 12, 14 through compressive forces applied through the load bearing element 20, the retention member 22, and the elongated fixation member 24. Additionally, the joint fixation system 10 provides adjustable tension between the facets 12, 14 based on the locking position of the elongated fixation member 24 with the retention member 22, and it is self-locking between the elongated fixation member 24 and the retention member 22. The joint fixation system 10 can be utilized with a bone graft or the like disposed between the facets 12, 14 to promote fusion or the like. Also, the joint fixation system 10 can be used to stabilize other joints besides the facet joint.

The elongated fixation member 24 includes a flexible and durable biocompatible material. For example, the elongated fixation member 24 can be constructed of stainless steel, cobalt-chromium alloy, titanium, Nitinol, ultra-high molecular weight polyethylene, cobalt-chromium alloy, poly(tetrafluoroethylene) or poly(tetrafluoroethene) (PTFE) or polyethylene terephthalate (PET). Optionally, the elongated fixation member 24 can be constructed of a radiolucent material, such as polyaryletheretherketone (PEEK) or the like, such that it can be medically visualized. The elongated fixation member 24 is flexible enough to be positioned within a hole in the facets 12, 14. An end 26 of the elongated fixation member 24 is fixedly attached, disposed, or connected to the retention member 22. The retention member 22 also includes a locking mechanism 28 and an opening 30. The retention member 22 includes a durable and rigid biocompatible material, such as stainless steel, cobalt-chromium alloy, titanium, Nitinol, ultra-high molecular weight polyethylene, cobalt-chromium alloy, PTFE or PET, PEEK, or the like. Another end 32 of the elongated fixation member 24 can be inserted into the opening 30. The end 32 of the elongated fixation member 24 is first inserted through each facet 12, 14 in the hole. This insertion can be accomplished by drilling a hole, by utilizing an insertion device such as a gun, or the like. The elongated fixation member 24 includes a flexible material to enable the elongated fixation member 24 to conform around and through the facets 12, 14, and the elongated fixation member 24 includes a strong and durable material to provide tension to hold the facets 12, 14 or other joint in place. The elongated fixation member 24 is of a length suitable to fit around the facets 12, 14 from the retention member 22 and back to through the opening 30 of the retention member 22. The elongated fixation member 24 can include a variety of lengths and dimensions as required for different spinal morphologies.

The guides 16, 18 enable routing of the elongated fixation member 24 through the hole in the facets 12, 14. The guides 16, 18 are utilized during deployment of the joint fixation system 10 and removed following locking of the elongated fixation member 24. The guide 18 is operable to route the elongated fixation member 24 to a hole in the facet 14, and the guide 16 is operable to route the elongated fixation member 24 from a hold in the facet 16 to the retention member 22. The guides 16, 18 can be integrated in a single surgical instrument with a handle (not shown) for use by the surgeon during deployment of the facet fixation system 10. This surgical instrument with the guides 16, 18 can be used in conjunction with a hole defining mechanism to form the hole in the facets 12, 14 to deploy the facet fixation system 10. During the engagement of the elongated fixation member 24, the load bearing element 20 is positioned between the facets 12, 14, the elongated fixation member 24, and the retention member 22. The load bearing element 20 is operable to move a load from the elongated fixation member 24 to a medial/lateral (M/L) direction to negate pull-out of the elongated fixation member 24 through the bone. The load bearing element 20 includes a flexible and durable material which is able to conform to the elongated fixation member 24 as it provides tension to the facets 12, 14 while allowing movement of the facet joint. Exemplary materials for the load bearing element 20 can include biocompatible material, such as stainless steel, cobalt-chromium alloy, titanium, Nitinol, ultra-high molecular weight polyethylene, cobalt-chromium alloy, PTFE or PET, PEEK, or the like.

After insertion of the lace through the facets 12, 14 and with the load bearing element 20 in place, the end 32 of the elongated fixation member 24 is positioned in the opening 30 of the retention member 22. The elongated fixation member 24 includes a plurality of teeth 34 which are disposed on or formed on the elongated fixation member 24 to provide a self-locking, i.e. “self-tying”, mechanism with the retention member 22. Alternatively, the plurality of teeth 34 could include a plurality of recessed valleys. The end 32 is selectively pulled through the opening 30 of the retention member 22 to a desired position for fixing the facets 12, 14, i.e. to provide enough tension or compression as required for facet joint fixation. At this point, one of the plurality of teeth 34 is operable to lock with a locking mechanism 28 in the retention member 22, providing self-locking/self-tying. The plurality of teeth 34 are include an angled shape that allows each of the plurality of teeth 34 to catch the latch 34 preventing the elongated fixation member 24 from backing out, but allowing the elongated fixation member 24 to be pulled further through the opening 30 thereby providing additional compression. The present invention also contemplates other locking mechanisms in addition to the plurality of teeth 34 as are known in the art. The locking mechanism 28 is located in the opening 30 of the retention member 22. Specifically, the opening 30 is dimensioned to receive both the ends 26, 28 of the elongated fixation member 24. The locking mechanism 28 can be attached, disposed, connected, or formed as a single piece with the retention member 22. For example, the locking mechanism 28 can include a latch operable to engage one of the teeth 34 or a defined notch or valley in the elongated fixation member 24. Additionally, the locking mechanism 28 includes a durable biocompatible material able to withstand pulling forces associated with the elongated fixation member 24. This self-locking mechanism includes the plurality of teeth 34 on the elongated fixation member 24 and the locking mechanism 28 in the retention member 22. Advantageously, the self-locking mechanism enables the joint fixation system 10 to lock a variable length by pulling the end 32 of the elongated fixation member 24 through the retention member 22 and leaving it once in the desired position. Once the elongated fixation member 24 is in the desired position, the tooth 34 that is captured by the locking mechanism 28 remains locked to the latch 28.

The hole in the facets 12, 14 or other joint can be formed through various mechanisms as are known in the art. An exemplary mechanism for forming the hole is described in commonly assigned U.S. patent application Ser. No. 12/395,300, filed Feb. 27, 2009, and entitled “FACET JOINT BROACHING INSTRUMENT, IMPLANT, AND ASSOCIATED METHOD,” the contents of which are herein incorporated by reference. The hole is formed in a desired location to provide stabilization/immobilization of the facet joint. Other mechanisms for forming the hole are also contemplated such as a drilling device or the like. Access to the facets 12, 14 can be gained through any suitable surgical technique and route using any suitable device or system. Advantageously, the joint fixation system 10 contemplates use with a minimally-invasive surgical (MIS) procedure.

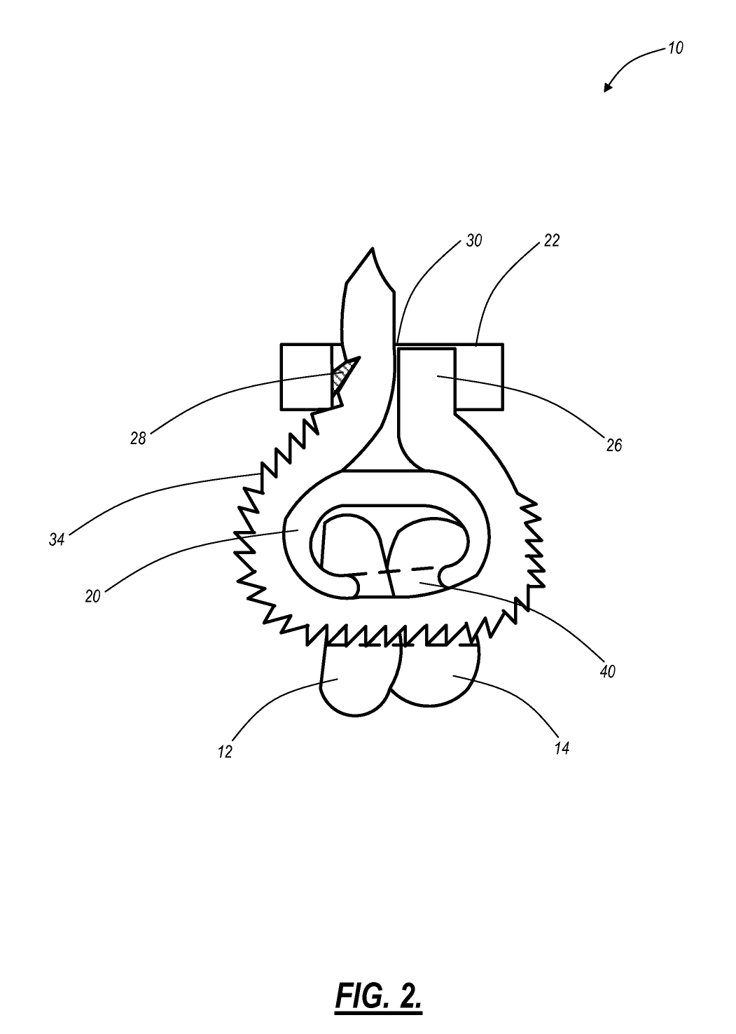

Referring to FIG. 2, the joint fixation system 10 for stabilizing facets 12, 14 of a facet joint is illustrated in a deployed configuration with the flexible elongated fixation member 24 tied to the retention member 22 according to an exemplary embodiment of the present invention. FIG. 2 illustrates the joint fixation system 10 after deployment and positioning of the elongated fixation member 24 by a surgeon. Here, the elongated fixation member 24 is shown inserted through a hole 40 in facets 12, 14 and through the retention member 22 opening 30, providing stabilization and immobilization of the joint associated with the facets 12, 14. The teeth 34 on the elongated fixation member 24 form a lock between the elongated fixation member 24 and the locking mechanism 28 in the retention member 22. Note that the end 28 on the elongated fixation member 24 can be cut or the like once the desired compression is determined, i.e. once the elongated fixation member 24 is pulled through the opening 30 to the desired location. Together, the teeth 34 and locking mechanism 28 can provide a ratchet action that allows a surgeon to provide a variable amount of tension to the facets 12, 14 based on the tightness of the elongated fixation member 24. Once set in the desired position, the teeth 34 and locking mechanism 28 hold the elongated fixation member 24 in position compressing the facets 12, 14 thereby fixing, immobilizing, and/or stabilizing the associated facet joint. The load bearing element 20 is operable to conform to a shape around the elongated fixation member 24 and the facets 12, 14 to negate the load in the M/L direction. Specifically, the load bearing element 20 is operable to engage both sides of the hole 40 and to be slightly disposed within the hole 40 with the elongated fixation member 24. This wrap-around configuration thereby negates the load on the facets 12, 14. As shown in FIG. 2, the joint fixation system 10 has fixed the facets 12, 14 together providing facet joint fixation.

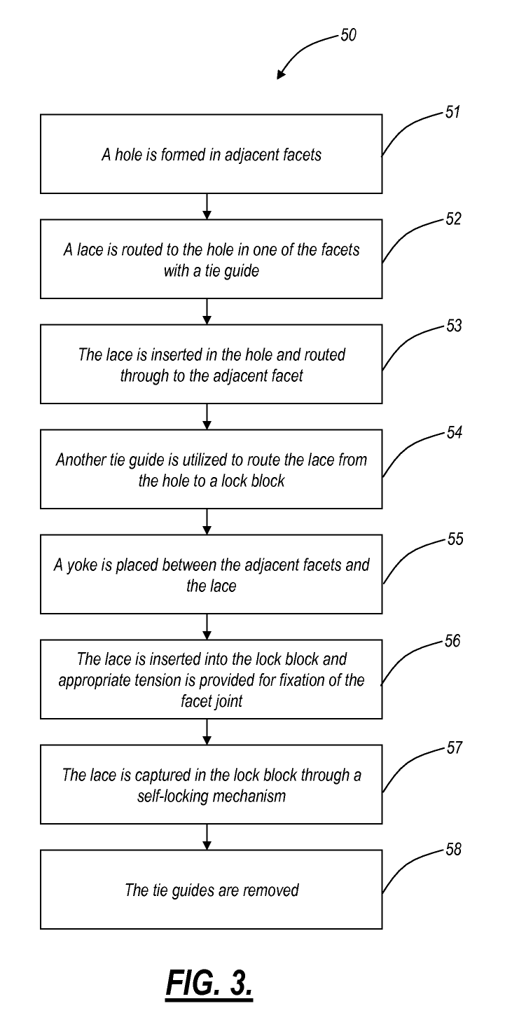

Referring to FIG. 3, a flowchart illustrates a mechanism 50 for fixing joints according to an exemplary embodiment of the present invention. The mechanism 50 can be utilized by a surgeon with the joint fixation system 10 of FIGS. 1 and 2 to provide joint fixation. Access is provided to a joint of a patient through various surgical techniques and routes. A hole is then formed in adjacent facets, i.e. a superior and inferior facet of a facet joint, or other joint (step 51). The hole can be formed through various mechanisms, and the hole is dimensioned to support an elongated fixation member and a load bearing element associated with the joint fixation system. The elongated fixation member is routed in the hole in one of the facets with a guide (step 52). The guide can be part of a surgical instrument that is operable to positioning the elongated fixation member to engage and route through the hole in the facets. The elongated fixation member is inserted in the hole and routed through to the adjacent facet (step 53).

Another guide is utilized to route the elongated fixation member from the hole exiting the adjacent facet to a retention member (step 54). The retention member is disposed to the elongated fixation member at one end. A load bearing element is placed between the adjacent facets and the elongated fixation member (step 55). The load bearing element is operable to negate a load in the M/L direction. The elongated fixation member is inserted into the retention member and appropriate tension is provided for fixation of the facet joint (step 56). Tension is provided by pulling an end of the elongated fixation member through the retention member. As tension is provided, the load bearing element conforms to both facets, and is slightly disposed in the hole in each facet. The elongated fixation member is captured in the retention member through a self-locking mechanism (step 57). The self-locking mechanism prevents the load bearing element from loosening, but allows the elongated fixation member to be tightened through the retention member. Finally, the guides are removed (step 58). The mechanism 50 is also contemplated for use with other joints.

Although the present invention has been illustrated and described herein with reference to preferred embodiments and specific examples thereof, it will be readily apparent to those of ordinary skill in the art that other embodiments and examples may perform similar functions and/or achieve like results. All such equivalent embodiments and examples are within the spirit and scope of the present invention and are intended to be covered by the following claims.

What is claimed is:

1. A surgical joint fixation system, comprising:
a retention mechanism defining an opening; and
an elongated fixation member comprising a first end and a second end, wherein the first end is disposed to the retention mechanism and the second end is selectively disposed through a hole in a joint and through the opening in the retention mechanism.

2. The surgical joint fixation system of claim 1, further comprising:
a locking mechanism between the retention mechanism and the elongated fixation member operable to lock the second end to the retention mechanism.

3. The surgical joint fixation system of claim 2, wherein the locking mechanism comprises a plurality of notches disposed along a portion of the length of the elongated fixation member and a latch disposed to the opening of the retention mechanism.

4. The surgical joint fixation system of claim 1, further comprising:
a load bearing element disposed between the joint and the elongated fixation member, wherein the load bearing element is operable to move a load formed by the elongated fixation member across the joint.

5. The surgical joint fixation system of claim 4, wherein the load bearing element wraps around the joint and engages an opening of the hole in the joint.

6. The surgical joint fixation system of claim 1, further comprising a guide operable to route the elongated fixation member from the retention mechanism to the hole and from the hole back to the opening of the retention mechanism.

7. The surgical joint fixation system of claim 6, wherein the guide is removed following insertion of the second end of the elongated fixation member into the opening of the retention mechanism.

8. The surgical joint fixation system of claim 1, wherein the elongated fixation member is pulled through the opening to provide a ratchet-like tension to the joint.

9. The surgical joint fixation system of claim 1, wherein a surgeon utilizes the surgical joint fixation system with a minimally invasive surgical procedure to provide immobilization and stabilization of a facet joint.

10. A facet joint fixation system, comprising:
a retention mechanism defining an opening with a locking device;
an elongated fixation member fixedly attached to the retention mechanism at one end of the elongated fixation member, wherein the elongated fixation member comprises a plurality of locking points substantially extending from the one end of the elongated fixation member to another end of the elongated fixation member, wherein the plurality of locking points are operable to lock with the locking device when the another end of the elongated fixation member is positioned and pulled through the opening of the retention mechanism;
a guide operable to guide the elongated fixation member to a hole through adjacent facets; and
a load bearing element placed between the elongated fixation member and the adjacent facets.

11. The facet joint fixation system of claim 10, wherein the guide is removed following insertion of the elongated fixation member into the opening of the retention mechanism.

12. The facet joint fixation system 10, wherein the elongated fixation member is pulled through the opening of the retention mechanism to provide a ratchet-like tension to the facets.

13. The facet joint fixation system of claim 10, wherein a surgeon utilizes the facet joint fixation system with a minimally invasive surgical procedure to provide immobilization and stabilization of the facet joint.

14. A joint fixation method, comprising:
forming a hole through a joint;
routing an elongated fixation member through the formed hole, wherein the elongated fixation member is fixedly attached to a retention member at one end;
routing the elongated fixation member from the formed hole back to the retention member;
inserting the elongated fixation member into an opening in the retention member, wherein the opening comprises a locking mechanism; and
adjusting the elongated fixation member to a tension for fixing the joint with the locking mechanism.

15. The joint fixation method of claim 14, wherein the routing steps utilize a guide to guide the elongated fixation member.

16. The joint fixation method of claim 14, further comprising placing a load bearing element between the elongated fixation member and the joint before adjusting the elongated fixation member.

17. The joint fixation method of claim 16, wherein the adjusting step further comprises forming the load bearing element to the joint and to the hole in the joint.

18. The joint fixation method of claim 14, wherein the adjusting step further comprises automatically locking of the elongated fixation member with the retention member through a ratchet mechanism.

19. The joint fixation method of claim 18, wherein the ratchet mechanism comprises the plurality of locks on the elongated fixation member each operable to lock to a lock on the retention member.
